(19) United States
(12) Patent Application Publication
(10) Pub. No.: US 2008/0090038 A1
(43) Pub. Date: Apr. 17, 2008

(54) Plastic product with a long-term dispersion of fragrance

(76) Inventor: Larry Yaw, Taichung (TW)

Correspondence Address: BRUCE H. TROXELL, SUITE 1404, 5205 LEESBURG PIKE, FALLS CHURCH, VA, 22041, US

(21) Appl. No.: 11/580,288

(22) Filed: Oct. 13, 2006

(51) Int. Cl. A61K 8/00

(52) U.S. Cl. 428/35.7

(57) ABSTRACT

A plastic product with a long-term dispersion of fragrance consists of a main body made of a plastic compound and plural aromatic master batches. The aromatic master batches are kept properly spaced apart in the main body to remain grainy as they are, able to keep dispersing fragrance for a long period of time.

### BACKGROUND OF THE INVENTION

1. Field of the Invention

This invention relates to a plastic product, particularly to one able to disperse fragrance in a long period of time without fading quickly.

2. Description of the Prior Art

Generally, all kinds of plastics have been tremendously demanded because they are so easily and conveniently applied in our life. And, with upgrading of living quality, plastic products are requested to have more additional values other than an exquisite appearance. To disperse fragrance is one of those additional values favored by people. A traditional manufacturing method of making a fragrant plastic product is commonly to mix aromatic master batches in plastic compound to form a mixture, which is then heated to become a melted liquid and, next, made by means of an injection or extrusion molding to form a product with aroma. But, the aromatic master batches are apt to be cracked to lose their fragrance during the making process of heating to melt together with the plastic compound, causing such a product having only a faint smell or unable to disperse aroma for a long period of time.

### SUMMARY OF THE INVENTION

The objective of this invention is to offer a plastic product with a long-term dispersion of fragrance.

The main characteristics of the invention are a main body and plural aromatic master batches. The aromatic master batches are put in the mold prior to molding the main body, so that the aromatic master batches remain as independent grainy individuals after being buried in the main body. Therefore, the aromatic master batches can maintain dispersing their fragrance for a long time, unlike the traditional ones heated to get cracked while manufacturing. Moreover, the aromatic master batches can be added with pigments or fluorescent agents to make them look more vividly.

### BRIEF DESCRIPTION OF DRAWINGS

This invention is better understood by referring to the accompanying drawings, wherein:

FIG. 1 is a perspective view of a preferred embodiment of a plastic product with a long-term dispersion of fragrance in the present invention; and

FIG. 2 is a cross-sectional view of the preferred embodiment of a plastic product with a long-term dispersion of fragrance in the present invention.

### DETAILED DESCRIPTION OF THE PREFERRED EMBODIMENT

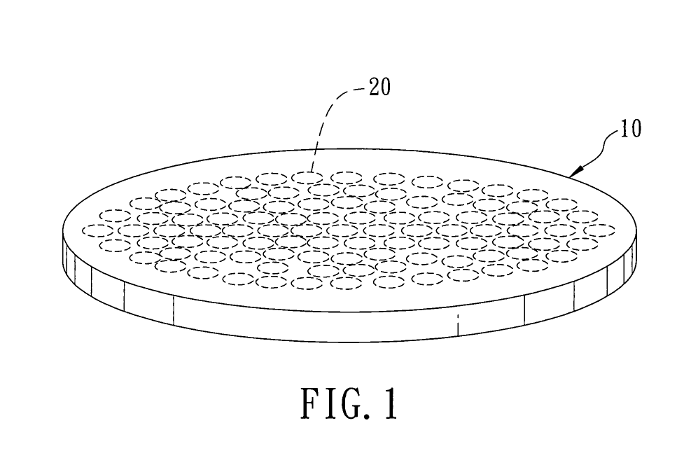

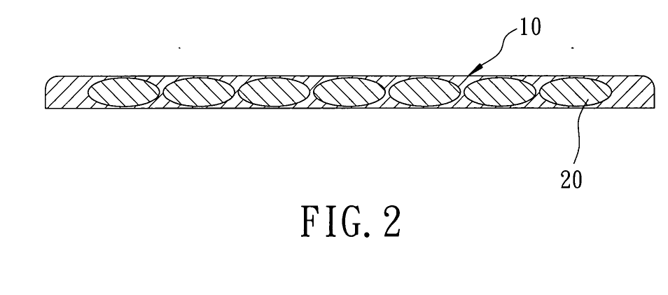

As shown in FIGS. 1 and 2, a preferred embodiment of a plastic product with a long-term dispersion of fragrance in the present invention includes a main body 10 and plural aromatic master batches 20.

The main body 10 made of plastics is an ornament.

The aromatic master batches 20 formed grainy are spread in and surrounded by the main body 10. The aromatic master batches 20 are placed properly spaced apart in the mold before the main body 10 is formed by injecting or dropping plastic compound in the mold, so that the aromatic master batches 20 can still be kept grainy after molding. In addition, the aromatic master batches 20 can be added with fluorescent agents or pigments previously so that they can be seen glaringly if buried in the main body 10 formed semi-transparent.

The invention has the following advantages as can be seen from the foresaid description.

1. The present invention can keep dispersing fragrance for a long period of time because the aromatic master batches 20 are set in the mold prior to molding the main body 10 so that they remain grainy as they are, able to prevent the aromatic master batches 20 from heated to crack to lose their smell as the traditional one does.

2. If the main body 10 is formed semi-transparent, the main body 10 and the aromatic master batches 20 can be added respectively with pigments or fluorescent agents to make them seen distinctively and dazzlingly, promoting aesthetic effect.

3. The present invention can be applied to various plastics to achieve economic effect.

While the preferred embodiment of the invention has been described above, it will be recognized and understood that various modifications may be made therein and the appended claims are intended to cover all such modifications that may fall within the spirit and scope of the invention.

What is claimed is:

1. A plastic product with a long-term dispersion of fragrance comprising:
a main body made of a plastic compound; and
plural aromatic master batches formed grainy and spread properly spaced apart inside said main body.

2. A plastic product with a long-term dispersion of fragrance as claimed in claim 1, wherein said aromatic master batches are added with fluorescent agents and pigments so that they can be seen distinctively through said main body.
